(19) United States
(12) Patent Application Publication
(10) Pub. No.: US 2002/0069433 A1
(43) Pub. Date: Jun. 6, 2002

(54) Apomixis conferred by expression of serk interacting proteins

(76) Inventors: Eduard Daniel Leendert Schmidt, Arnhem (NL); Sape Cornelis De Vries, Wageningen (NL); Valerie France Gabrielle Hecht, Wageningen (NL)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 09/839,185

(22) Filed: Apr. 19, 2001

Related U.S. Application Data
(63) Continuation of application No. PCT/EP99/07972, filed on Oct. 20, 1999.

(30) Foreign Application Priority Data
Oct. 22, 1998 (GB) 9823098.0

(51) Int. Cl. A01H 5/00; C12N 15/82

(52) U.S. Cl. 800/298; 800/278; 435/419; 536/23.6

(57) ABSTRACT

The present invention relates to a method for increasing the probability of vegetative reproduction of a new plant generation by transgenic expression of a gene encoding a protein acting in the signal transduction cascade triggered by the Somatic Embryogenesis Receptor Kinase (SERK). Apomictic seeds resulting therefrom, plants and progeny obtained through germination of such seeds, and genes encoding proteins acting in the signal transduction cascade triggered by SERK constitute further subject matters of the invention.

The present invention relates to vegetative reproduction of plants and plant cells. In particular the invention relates to a method for increasing the probability of vegetative reproduction in vivo through seeds or in vitro by somatic embryogenesis. Apomictic seeds resulting therefrom, and the plants and progeny obtained through germination of such seeds are further subject matters of the invention.

Vegetative, non-sexual reproduction through seeds also called apomixis, is a genetically controlled reproductive mechanism of plants found in some polyploid non-cultivated species. Two types of apomixis, gametophytic or non-gametophytic, can be distinguished. In gametophytic apomixis —of which there are two types, namely apospory and diplospory —multiple embryo sacs typically lacking antipodal nuclei are formed, or else megasporogenesis in the embryo sac takes place. In non-gametophytic apomixis also called adventitious embryony, a somatic embryo develops directly from the cells of the embryo sac, ovary wall or integuments. Somatic embryos from surrounding cells invade the sexual ovary, one of the somatic embryos out-competes the other somatic embryos and the sexual embryo, and utilizes the produced endosperm.

Engineering apomixis to a controllable, more reproducible trait would provide many advantages in plant improvement and cultvar development in case that sexual plants are available as crosses with the apomictic plant. The Somatic Embryogenesis Receptor Kinase (SERK) is known to be involved in the formation of extraneous embryos from sporophytic cells which can result in apomictic seeds.

Apomixis would provide for true-breeding, seed propagated hybrids. Moreover, apomixis could shorten and simplify the breeding process so that selfing and progeny testing to produce and/or stabilize a desirable gene combination could be eliminated. Apomixis would provide for the use as cultivars of genotypes with unique gene combinations since apomictic genotypes breed true irrespective of heterozygosity. Genes or groups of genes could thus be “pyramided and “fixed” in super genotypes. Every superior apomictic genotype from a sexual-apomictic cross would have the potential to be a cultivar. Apomixis would allow plant breeders to develop cultivars with specific stable traits for such characters as height, seed and forage quality and maturity.

Breeders would not be limited in their commercial production of hybrids by (i) a cytoplasmic-nuclear interaction to produce male sterile female parents or (ii) the fertility restoring capacity of a pollinator. Almost all cross-compatible germplasm could be a potential parent to produce apomictic hybrids.

Apomixis would also simplify commercial hybrid seed production. In particular, (i) the need for physical isolation of commercial hybrid production fields would be eliminated; (ii) all available land could be used to increase hybrid seed instead of dividing space between pollinators and male sterile lines; and (iii) the need to maintain parental line seed stocks would be eliminated.

The potential benefits to accrue from the production of seed via apomixis are presently unrealized, to a large extent because of the problem of engineering apomictic capacity into plants of interest. The present invention teaching introduction of proteins acting in the signal transduction cascade triggered by SERK provides a further step to the solution of that problem in that it improves vegetative reproduction in vivo through seeds and in vitro by somatic embryogenesis.

In the following the term “gene” refers to a coding sequence and associated regulatory sequences. The coding sequence is transcribed into RNA, which depending on the specific gene, will be mRNA, rRNA, tRNA, snRNA, sense RNA or antisense RNA. Examples of regulatory sequences are promoter sequences, 5′ and 3′ untranslated sequences and termination sequences. Further elements that may be present are, for example, introns.

A “promoter” is a DNA sequence initiating transcription of an associated DNA sequence. Depending on the specific promoter region it may also include elements that act as regulators of gene expression such as activators, enhancers, and/or repressors.

A regulatory DNA sequence such as promoter is said to be “operably linked to” or “associated with” a DNA sequence that codes for an RNA or a protein, if the two sequences are situated such that the regulatory DNA sequence affects expression of the coding DNA sequence.

The term “expression” refers to the transcription and/or translation of an endogenous gene or a transgene in plants.

Expression “in the vicinity of the embryo sac” is considered to mean expression in carpel, integuments, ovule, ovule premordium, ovary wall, chalaza, nucellus, funicle or placenta. The skilled man will recognize that the term “integuments” can include tissues which are derived therefrom, such as endothelium. “Embryogenic” defines the capability of cells to develop into an embryo under permissive conditions. It will appreciated that the term “in an active form” includes proteins which are truncated or otherwise mutated with the proviso that they still increase the probability of vegetative reproduction whether or not in doing this they interact with the signal transduction components that they otherwise would in the tissues in which they are normally present.

“Marker genes” encode a selectable or screenable trait. Thus, expression of a “selectable marker gene” gives the cell a selective advantage which may be due to their ability to grow in the presence of a negative selective agent, such as an antibiotic or a herbicide compared to the growth of non-transformed cells. The selective advantage possessed by the transformed cells, compared to non-transformed cells, may also be due to their enhanced or novel capacity to utilize an added compound as a nutrient, growth factor or energy source. Selectable marker gene also refers to a gene or a combination of genes whose expression in a plant cell gives the cell both, a negative and a positive selective advantage. On the other hand a “screenable marker gene” does not confer a selective advantage to a transformed cell, but its expression makes the transformed cell phenotypically distinct from untransformed cells.

The term “plant” refers to any plant, but particularly seed plants.

The term “plant cell” describes the structural and physiological unit of the plant, and comprises a protoplast and a cell wall. The plant cell may be in form of an isolated single cell (such as a stomatal guard cells) or a cultured cell, or as a part of a higher organized unit such as, for example, a plant tissue, or a plant organ.

The term “plant material” includes leaves, stems, roots, emerged radicles, flowers or flower parts, petals, fruits, pollen, pollen tubes, anther filaments, ovules, embryo sacs, egg cells, ovaries, zygotes, embryos, zygotic embryos per se, somatic embryos, hypocotyl sections, apical meristems, vascular bundles, pericycles, seeds, cuttings, cell or tissue cultures, or any other part or product of a plant

The following solutions are provided by the present invention:

A method for increasing the probability of vegetative reproduction of a new plant generation comprising transgenically expressing a gene encoding a protein acting in the signal transduction cascade triggered by the Somatic Embryogenesis Receptor Kinase (SERK);

said method wherein the encoded protein physically interacts with SERK;

said method wherein the protein is a member of the family of Squamosa-promoter Binding Protein (SBP) transcription factors or 14-3-3 type lambda proteins;

said method wherein the protein has the amino acid sequence given in SEQ ID NO:2, SEQ ID NO:4, SEQ ID NO:6, SEQ ID NO:8, SEQ ID NO:10, SEQ ID NO:12, SEQ ID NO:14, or SEQ ID NO:16, or an amino acid sequence having a component sequence of at least 150 amino acids length which after alignment reveals at least 40% identity with SEQ ID NO:12 or SEQ ID NO:16;

said method increasing the probability of vegetative reproduction through seeds (apomixis);

said method wherein the seeds result from non-gametophytic apomixis;

said method wherein the encoded protein is transgenically expressed in the vicinity of the embryo sac;

said method increasing the probability of in vitro somatic embryogenesis;

said method wherein expression of the gene is under control of the SERK gene promoter, the carrot chitinase DcEP3-1 gene promoter, the Arabidopsis AtChitIV gene promoter, The Arabidopsis LTP-1 gene promoter, The Arabidopsis bel1 gene promoter, the petunia fbp-7 gene promoter, the Arabidopsis ANT gene promoter or the promoter of the O 126 gene of Phalaenopsis;

a gene encoding a protein having the amino acid sequence given in SEQ ID NO:2, SEQ ID NO:4, SEQ ID NO:6, SEQ ID NO:8, SEQ ID NO:10, SEQ ID NO:12, SEQ ID NO: 14, or SEQ ID NO:16, or an amino acid sequence having a component sequence of at least 150 amino acids length which after alignment reveals at least 40% sequence identity with SEQ ID NO:12 or SEQ ID NO:16;

said gene having the nucleotide given in SEQ ID NO:1, SEQ ID NO:3, SEQ ID NO:5, SEQ ID NO:7, SEQ ID NO:9,SEQ ID NO: 11, SEQ ID NO:13, or SEQ ID NO: 15;

said gene wherein the nucleotide sequence is modified in that known mRNA instability motifs or polyadenylation signals are removed and/or condons which are preferred by the plant into which the DNA is to be inserted are used;

a plant or plant cell transgenically expressing said gene; and

a plant or plant cell obtainable by the method according to the present invention.

Another to the present invention there is provided a method for increasing the probability of vegetative reproduction of a new plant generation, for example by producing apomictic seeds or generating somatic embryos under in vitro conditions, comprising transgenically expressing a gene encoding a protein acting in the signal transduction cascade triggered by the Somatic Embryogenesis Receptor Kinase (SERK). This is achieved by (i) transforming plant material with a nucleotide sequence encoding said protein (ii) regenerating transformed plant material into plants, or carpel-containing parts thereof, and (iii) expressing the sequence in the vicinity of the embryo sac.

A further embodiment of the invention relates to genes encoding proteins acting in the signal transduction cascade triggered by the Somatic Embryogenesis Receptor Kinase (SERK) the presence of which in an active form in a cell, or membrane thereof, renders said cell embryogenic.

The gene to be expressed preferably encodes a protein physically interacting with SERK. Specific examples of SERK-interacting proteins are members of the family of Squamosa-promoter Binding Protein (SBP) transcription factors (Klein et al, Mol Gen Genet 250:7-16, 1996). These proteins are able to interact specifically with DNA through a conserved domain of 70 to 90, perferably 79 amino acid residues, the SBP-box. Alignment of different SBP-box sequences generally reveals at least 50% and preferably more than 60% or more than 70% sequence identity. Within the SBP-box remarkable arrangement of cysteine and histidine residues can be recognized, which is reminiscent of zinc-fingers and probably involved in the recognition of specific promoter elements. A bipartite nuclear localization signal is placed at the C-terminal end of the SBP-box (Dingwall et al, Trends Biochem Sci 16:478-481, 1991). Both the N-terminal and the C-terminal domains of the SERK-interacting SBP proteins are highly variable and are probably involved in regulation of protein activity. One of the possible SBP proteins is identical with SPL3 (SEQ ID NO:5 and SEQ ID NO:6), a gene involved in the floral transition and expressed in developing flower buds (Cardon et al, Plant Journal 12:367-377, 1997).

Another class of SERK-interacting proteins are isoforms of the family of 14-3-3 proteins such as the 14-3-3 type lambda proteins (Wu et al, Plant Physiol 114:1421-1431, 1997; SEQ ID NO:9 and SEQ ID NO:10). A total of 10 different 14-3-3 proteins are present in Arabidopsis the different members being involved in intracellular signal transduction. They mediate signal transduction by binding to phosphoserine-containing proteins on specific binding motifs represented by conserved amino acid sequences like RxxS(p)xP (Yaffe et al, Cell 91:961-971, 1997). A putative 14-3-3 interaction domain having the amino acid sequence RPPSQP is also found at position 391-396 of the Arabidopsis SERK protein, and at the corresponding aligned region of the Daucus carota SERK protein having the amino acid sequence RQPSEP providing SERK with a mechanism for a 14-3-3 mediated signal transduction.

A further class of SERK-interacting proteins is exemplified by SEQ ID NO:11 (and SEQ ID NO:12) and the NDR1 protein already described in the literature (Century et al, Science 278:1963-1965, 1997). NDR1 is likely to encode a membrane-associated component in the signal transduction pathway downstream of pathogen-recognizing proteins. It was suggested that NDR1 might be a protein that interacts with many different receptors. SEQ ID NO:6 represents a new member in this small family of proteins supposed to function in intracellular signal transduction mediated by transmembrane receptors.

SEQ ID NO:13 encodes a SERK-interaction protein (SEQ ID NO:14) with homology to a domain of E.coli aminopeptidase N and is expected to encode an Arabidopsis protease interacting with or activated by SERK.

The predicted amino acid sequence of the SERK-interacting protein of SEQ ID NO:15 (SEQ ID NO:16) has no homology with known gene products although there is a small not yet described family of related gene products in Arabidopsis.

Insofar as the the SERK-interacting proteins mentioned above and their corresponding genes are novel they constitute a further subject matter of the present invention.

Of course, genes similar to the ones described above can also be used. A similar gene is a gene having a nucleotide sequence complementary to the test sequence and capable of hybridizing to the inventive sequence. When the test and inventive sequences are double stranded the nucleic acid constituting the test sequence preferably has a TM within 20° C. of that of the inventive sequence. In the case that the test and inventive sequences are mixed together and denatured simultaneously, the TM values of the sequences are preferably within 10° C. of each other. More preferably the hybridization is performed under stringent conditions, with either the test or inventive DNA preferably being supported. Thus either a denatured test or inventive sequence is preferably first bound to a support and hybridization is effected for a specified period of time at a temperature of between 50° and 70° C. in double strength citrate buffered saline (SSC) containing 0.1% SDS followed by rinsing of the support at the same temperature but with a buffer having a reduced SSC concentration. Depending upon the degree of stringency required, and thus the degree of similarity of the sequences, at a particular temperature, —such as 60° C. for example —such reduced concentration buffers are typically single strength SSC containing 0.1% SDS, half strength SSC containing 0.1% SDS and one tenth strength SSC containing 0.1% SDS. Sequences having the greatest degree of similarity are those the hybridization of which is least affected by washing in buffers of reduced concentration. It is most preferred that the test and inventive sequences are so similar that the hybridization between them is substantially unaffected by washing or incubation in one tenth strength sodium citrate buffer containing 0.1% SDS.

The gene to be expressed may be modified in that known mRNA instability motifs or polyadenylation signals are removed or codons which are preferred by the plant into which the sequence is to be inserted may be used so that expression of the thus modified sequence in the said plant may yield substantially similar protein to that obtained by expression of the unmodified sequence in the organism in which the protein is endogenous.

The sequence variability of proteins with similar function suggests, that a number of amino acids can be replaced, inserted or deleted without altering a protein's function. The relationship between proteins is reflected by the degree of sequence identity between aligned amino acid sequences of individual proteins or aligned component sequences thereof.

Dynamic programming algorithms yield different kinds of alignments. In general there exist two approaches towards sequence alignment. Algorithms as proposed by Needleman and Wunsch and by Sellers align the entire length of two sequences providing a global alingment of the sequences. The Smith-Waterman algorithm on the other hand yields local alignments. A local alignment aligns the pair of regions within the sequences that are most similiar given the choice of scoring matrix and gap penalties. This allows a database search to focus on the most highly conserved regions of the sequences. It also allows similiar domains within sequences to be identified. To speed up alignments using the Smith-Waterman algorithm both BLAST (Basic Local Alignment Search Tool) and FASTA place additional restrictions on the alignments.

Within the context of the present invention alignments are conveniently performed using BLAST, a set of similarity search programs designed to explore all of the available sequence databases regardless of whether the query is protein or DNA. Version BLAST 2.0 (Gapped BLAST) of this search tool has been made publicly available on the internet (currently http://www.ncbi.nlm.nih.gov/BLAST). It uses a heuristic algorithm which seeks local as opposed to global alignments and is therefore able to detect relationships among sequences which share only isolated regions. The scores assigned in a BLAST search have a well-defined statistical interpretation. Particularly useful within the scope of the present invention are the blastp program allowing for the introduction of gaps in the local sequence alignments and the PSI-BLAST program, both programs comparing an amino acid query sequence against a protein sequence database, as well as a blastp variant program allowing local alignment of two sequences only. Said programs are preferably run with optional parameters set to the default values.

Sequence alignments using BLAST can also take into account whether the substitution of one amino acid for another is likely to conserve the physical and chemical properties necessary to maintain the structure and function of a protein or is more likely to disrupt essential structural and functional features. For example non-conservative replacements may occur at a low frequency and conservative replacements may be made between amino acids within the following groups:

(i) Serine and Threonine;

(ii) Glutamic acid and Aspartic acid;

(iii) Arginine and Lysine;

(iv) Asparagine and Glutamine;

(v) Isoleucine, Leucine, Valine and Methionine;

(vi) Phenylatanine, Tyrosine and Tryptophan

(vii) Alanine and Glycine.

Such sequence similarity is quantifies in terms of percentage of positive amino acids, as compared to the percentage of identical amino acids and can help assigning a protein to the correct protein family in border-line cases.

Specific embodiments of the invention express a gene comprising a DNA sequence encoding a protein acting in the signal transduction cascade triggered by the Somatic Embryogenesis Receptor Kinase (SERK) and having the amino acid sequence depicted in SEQ ID NO:2, 4, 6 or 8, or a protein similar thereto. By similar is meant a protein having a component sequence of at least 150 amino acids length which after alignment reveals at least 40% and preferably 50% or more sequence identity with another protein.

In order to obtain expression of the sequence in a regenerated plant and in particular the carpel thereof in a tissue specific manner the sequence is under expression control of an inducible or developmentally regulated promoter. It is preferred that the gene is expressed in the somatic cells of the embryo sac, ovary wall, nucellus, or integuments. As the endosperm within the apomictic seed results from fusion of polar nuclei within the embryo sac with a pollen-derived male gamete nucleus it is preferred that the sequence encoding the protein is expressed prior to fusion of the polar nuclei with the male gamete nucleus.

Typically promoters are promoter which regulated expression of SERK genes in planta, the Arabidopsos ANT gene promoter, the promoter of the O126 gene from Phalaenopsis, the carrot chitinase DcEp3-1 gene promoter, the Arabidopsis AtChitIV gene promoter, the Arabidopsis LTP-1 gene promoter, the Arabidopsis bel-1 gene promoter, the petunia fbp-7 and fbp-11 gene promoters, the Arabidopsis AtDMC1 promoter, the pTA7001 inducible promoter. The DcEp3-1 gene is expressed transiently during inner integument degradation and later in cells that line the inner part of the developing endosperm. The AtChiIV gene is transiently expressed in the micropylar endosperm up to cellularisation. The LTP-1 promoter is active in the epidermis of the developing nucellus, both integuments, seed coat and early embryo. The bel-1 gene is expressed in the developing inner integument and the fbp-7 promoter is active during embryo sac development. The Arabidopsis ANT gene is expressed during integument development, and the O126 gene from Phalaenopsis is expressed in the mature ovule.

The promoters of the DcEP3-1 and the AtChit IV genes may be cloned and characterized by standard procedures. The gene encoding a protein of the SERK signal cascade is cloned behind the DcEp3-1 , the AtChit IV or the AtLTP-1 promoters and transformed into Arabidopsis. The ligation is performed in such a way that the promoter is operably linked to the sequence to be transcribed. This construct, which also contains known marker genes providing for selection of transformed material, is inserted into the T-DNA region of a binary vector such as pBIN19 and transformed into Arabidopsis. Agrobacterium-mediated transformation into Arabidopsis is performed by the vacuum infiltration or root transformation procedures known to the skilled man. Transformed seeds are selected and harvested and (where possible) transformed lines are established by normal selfing. Parallel transformations with 35S promoter constructs and the entire SERK-interacting gene itself are used as controls to evaluate over-expression in many cells or only in the few cells that naturally express the gene. The 35S promoter construct may give embryo formation wherever the signal that activates SERK-mediated transduction is present in the plant. A testing system based on emasculation and the generation of donor plant lines for pollen carrying LTP1 promoter-GUS and SERK promoter-bamase is established.

The same constructs (35S, EP3-1, AtChitIV, AtLTP-1 and SERK promoters fused to SERK-interacting coding sequences) can be employed for transformation into several Arabidopsis backgrounds such as wild type, male sterile, fis (allelic to emb 173) and primordia timing (pt)-1 lines, or a combination of two or several of these backgrounds. The wt lines are used as a control to evaluate possible effects on normal zygotic embryogenesis, and to score for seed set without fertilization after emasculation. The ms lines are used to score directly for seed set without fertilization. The fis lines exhibit a certain degree of seed and embryo development without fertilization, so may be expected to have a natural tendency for apomictic embryogenesis, which may be enhanced by the presence of the constructs. The pt-1 line has superior regenerative capabilities and has been used to initiate the first stably embryogenic Arabidopsis cell suspension cultures. Combinations of several of the above backgrounds are obtained by crossing with each other and with lines expressing SERK-interacting proteins ectopically. Except for the ms lines, propagation can proceed by normal selfing, and analysis of apomictic traits. A similar strategy is followed if the ATChiIV, AtLTP-1 and SERK promoters are replaced by the bel-1 and fbp-7 promoters as well by other promoters specific for components of the female gametophyte.

The invention still further includes vectors comprising DNA as indicated in the preceding paragraphs, plants transformed with the vector, progeny of such plants which contain the DNA stably incorporated, and the apomictic seeds of such plants or such progeny.

The genes to be expressed can be introduced into the plant cells in a number of art-recognized ways summarized in the paragraph bridging pages 7 and 8 of WO 97/43427.

Comprised within the scope of the present invention are transgenic plants, in particular transgenic fertile plants transformed by means of the aforedescribed processes and their asexual and/or sexual progeny, which still contain the DNA stably incorporated, and/or the apomictic seeds of such plants or such progeny. Said plants can be used in the same way as described on pages 10 to 12 of WO 97/43427.

A transgenic plant according to the invention may be a dicotyledonous or a monocotyledonous plant. Such plants include field crops, vegetables and fruits including tomato, pepper, melon, lettuce, cauliflower, broccoli, cabbage, brussels sprout, sugar beet, corn, sweetcorn, onion, carrot, leek, cucumber, tobacco, alfalfa, aubergine, beet, broad bean, celery, chicory, cow pea, endive, gourd, groundnut, papaya, pea, peanut, pineapple, potato, safflower, snap bean, soybean, spinach, squashes, sunflower, sorghum, water-melon, and the like; and ornamental crops including Impatiens, Begonia, Petunia, Pelargonium, Viola, Cyclamen, Verbena, Vinca, Tagetes, Primula, Saint Paulia, Ageratum, Amaranthus, Anthirrhinum, Aquilegia, Chrysanthemum, Cineraria, Clover, Cosmo, Cowpea, Dahlia, Datura, Delphinium, Gerbera, Gladiolus, Gloxinia, Hippeastrum, Mesembryanthemum, Salpiglossis, Zinnia, and the like. In a preferred embodiment, the DNA is expressed in “seed crops” such as corn sweet corn and peas etc. in such a way that the apomictic seed which results from such expression is not physically mutated or otherwise damaged in comparison with seed from untransformed like crops. Preferred are monocotyledonous plants of the Gramtnaceae family involving Lolium, Zea, Triticum, Trticale, Sorghum, Saccharm, Bromus, Oryzae, Avena, Hordeum, Secale and Setaria plants.

More preferred are transgenic maize, wheat, barley, sorghum, rye, oats, turf and forage grasses, millet, rice and sugar cane. Especially preferred are maize, wheat, sorghum, rye, oats, turf grasses and rice.

Among the dicotyledonous plants Arabidopsis, soybean, cotton, sugar beet, oilseed rape, tobacco and sunflower are more preferred herein. Especially preferred are tomato, pepper, melon lettuce, Brassica vegetables, soybean, cotton, tobacco, sugar beet and oilseed rape.

The expression ‘progeny’ is understood to embrace both, “asexually” and “sexually” generated progeny of transgenic plants. This definition is also meant to include all mutants and variants obtainable by means of known processes, such as for example cell fusion or mutant selection and which still exhibit the characteristic properties of the initial transformed plant, together with all crossing and fusion products of the transformed plant material. This also includes progeny plants that result from a backcrossing, as long as the said progeny plants still contain the DNA according to the invention.

Another object of the invention concerns proliferation material of the transgenic plants. It is defined relative to the invention as any plant material that may be propagated sexually or asexually in vivo or in vitro. Particularly preferred within the scope of the present invention are protoplasts, cells, calli, tissues, organs, seeds, embryos, pollen, egg cells, zygotes, together with any other propagating material obtained from transgenic plants. Parts of plants, such as for example flowers, stems, fruits, leaves, roots originating in transgenic plants or their progeny previously transformed by means of the process of the invention and therefore consisting at least in part of transgenic cells, are also an object of the present invention. Especially preferred are apomictic seeds.

The present invention is examplified by transgenic expression of a SERK-interacting gene in Arabidopsis under the control of plant expression signals, particularly a promoter which regulates expression of SERK genes in planta, but preferably a developmentally regulated or inducible promoter such as, for example, the carrot chitinase DcEp3-1 gene promoter, the Arabidopsis AtChitIV gene promoter, the Arabidopsis LTP-1 gene promoter, the Arabidopsis bel-1 gene promoter, the petunia fbp-7 gene promoter, the Arabidopsis ANT gene promoter, or the promoter of the O126gene from Phalaenopsis; the Arabidopsis AtDMC1 promoter, or the pTA7001 inducible promoter.

The promoters of the DcEP3-1 and the AtChit IV genes may be cloned and characterized by standard procedures. The desired coding sequence is cloned behind the DcEP3-1, the AtChit IV or the AtLTP-1 promoters and transformed into Arabidopsis. The ligation is performed in such a way that the promoter is operably linked to the sequence to be transcribed. This construct, which also contains known marker genes providing for selection of transformed material, is inserted into the T-DNA region of a binary vector such as pBIN19 and transformed into Arabidopsis. Agrobacterium-mediated transformation into Arabidopsis is performed by the vacuum infiltration or root transformation procedures known to the skilled man. Transformed seeds are selected and harvested and (where possible) transformed lines are established by normal selfing. Parallel transformations with 35S promoter constructs and the entire SERK-interacting gene itself are used as controls to evaluate over-expression in many cells or only in the few cells that naturally express the gene. The 35S promoter construct may give embryo formation wherever the signal that activates SERK-mediated transduction is present in the plant. A testing system based on emasculation and the generation of donor plant lines for pollen carrying LTP1 promoter-GUS and SERK promoter-bamase is established.

The same constructs (35S, EP3-1, AtCHitIV, AtLTP-1 and SERK promoters fused to the SERK-interacting coding sequence) are employed for transformation into several Arabidopsis backgrounds. These backgrounds are wild type, male sterile, fis (allelic to emb 173) and primordia timing (pt)-1 lines, or a combination of two or several of these backgrounds. The wt lines are used as a control to evaluate possible effects on normal zygotic embryogenesis, and to score for seed set without fertilization after emasculation. The ms lines are used to score directly for seed set without fertilization. The fis lines exhibit a certain degree of seed and embryo development without fertilization, so may be expected to have a natural tendency for apomictic embryogenesis, which may be enhanced by the presence of the constructs. The pt-1 line has superior regenerative capabilities and has been used to initiate the first stably embryogenic Arabidopsis cell suspension cultures. Combinations of several of the above backgrounds are obtained by crossing with each other and with lines expressing SERK-interacting proteins ectopically. Except for the ms lines, propagation can proceed by normal selfing, and analysis of apomictic traits. A similar strategy is followed in which the ATChiIV, AtLTP-1 and SERK promoters are replaced by the bel-1 and fbp-7 promoters as well by other promoters specific for components of the female gametophyte.

Whilst the present invention has been particularly described by way of the production of apomictic seed by heterologous expression of a SERK-interacting gene in the nucellar region of the carpel, the skilled man will recognize that other genes, the products of which have as similar structure/function may likewise be expressed with similar results. Moreover, although the example illustrates apomictic seed production in Arabidopsis, the invention is, of course, not limited to the expression of apomictic seed-inducing genes solely in this plant. Moreover, the present disclosure also includes the possibility of expressing the inventive gene sequences in transformed plant material in a constitutive, tissue non-specific manner, for example under transcriptional control of a CaMV35S or NOS promoter.

The skilled man who has the benefit of the present disclosure will also recognize that a SERK-interacting genes may be transformed into plant material which may be propagated and/or differentiated and used as an explant from which somatic embryos can be obtained. Expression of such sequences in the transformed tissue substantially increases the percentage of the cells in the tissue which are competent to form somatic embryos, in comparison with the number present in non-transformed like tissue.

The following examples illustrate the isolation and cloning of genes encoding SERK-interacting proteins and the production of apomictic seed by heterologous expression of said genes in the nucellar region of the carpel so that somatic embryos form which penetrate the embryo sac and are encapsulated by the seed as it develops.

### EXAMPLES

### Example 1

### Isolation of Arabidopsis Genes Endocing Proteins Interacting with the Arabidopsis SERK Gene roduct p

Construction of a SERK bait plasmid

The cDNA sequence of Arabidopsis SERK clone AtSERKtot61in pBluescript SK- is used as the DNA template to amplify by PCR the SERK open reading frame devoid of its N-terminal sequence using the oligonucleotide primers

V6 (5′-ATGCTTTGCATAACTTTGAGG-3′; SEQ ID NO:17) and

T7 (5′-AATACGACTCACTATAG-3′; SEQ ID NO:18).

The resulting PCR product is cloned into the vector pGEM-T (Promega). From the resulting plasmid an Ncol-Notl fragment is isolated and cloned into the Ncol-Notl sites of the yeast lexA two hybrid bait vector pEG202 SERK (Origene). Nucleotide sequence analysis is performed to confirm the correct orientation and sequence of the PCR product in the resulting SERK bait plasmid. Bait protein expression and activity is determined using along the protocols described in Current Protocols in Molecular Biology 1996, chapter 20, supplement 33, contributed by E. A. Golemis; J. Gyuris and R. Brent. The construct is shown to possess transcriptional activity in yeast strain EGY48. Furthermore, repressor activity on a reporter gene shows correct nuclear localization of the SERK gene product. Yeast transformed with the SERK bait plasmid proves to be leucine heterotrophic, indicating that the construct is not resulting in autoactivation of the lexA selection screen. The tests demonstrate that the SERK bait construct is suitable for lexA two hybrid screening.

Screening of a lexA two hybrid library

Yeast strain EGY48 transformed with the LacZ reporter plasmid pSH18-34 (Origene) and the bait vector pEG202 SERK is transformed with the cDNA library vector pJG4-5 (Origene) according to the LiAc/PEG4000 procedure described in Current protocols in Molecular Biology 1996, chapter 20, supplement 33, contributed by E. A. Golemis; J. Gyuris and R. Brent. A cDNA library from Arabidopsis thanliana young silique tissue containing early globular stage embryos is obtained (provided by Prof. Gerd Jürgens, Tuebingen). The primary library contains approximately 2,000,000 cDNA clones and the average insert length is 1.4 kB (as calculated from 90 clones of which the insert length caries from 0.2 to 4.5 kB). 10% of the clones contain no insert. The library is amplifies once in E.coli before screening for SERK protein interaction. Induction of the fusion proteins in pJG4-5 is by the application of galactose in the medium. Under non-inducing conditions, yeast cells are grown in glucose and do not express the pJG4-5 fusion proteins. 4,200,000 prey cDNA clones are transformed into the yeast strain containing the pEG202 SERK bait plasmid and the pSH18-34 reporter plasmid. Transformation efficiency is up to 270,000 colonies per microgram of vector DNA. The plasmid pJG4-5 contains the TRP1 selectable marker, pSH18-34 has an URA3 selectable marker and pEG202 contains a HIS3 selectable marker. Growth of the transformed yeast cells is taking place in complete minimal (CM) medium supplemented with either 2% glucose or 2% galactose+raffinose (in the latter case the galactose-inducible promoter on the vector pJG4-5 is activated, resulting in expression of the cDNA library fusion proteins. Yeast strain EGY48 contains six LexA operators which direct transcription from the LEU2 gene. When both the SERK fusion protein and the cDNA library fusion protein are expressed the LexA DNA-binding domain of the SERK fusion protein can interact with the activation domain of the library cDNA fusion protein to form an active LexA transcription factor which in turn allows to select for leucine autotropic transformants. The LacZ reporter construct on the plasmid pSH18-34 contains one LexA operator in a promoter context different from the LEU2 gene. Xgal and the presence of an active LexA transcription complex also allows determination of LacZ activity.

Triple selection for all three plasmids is performed on GLU/Cm-his-ura-trp 24 cm/24 cm plates with approximately 100,000 colonies per plate. A total of 4.200.000 yeast primary transformants are obtained. The colonies are scraped from the plates with a sterile glass slide, collected in two different A or B labeled 50 ml tubes and frozen −80° C. In order to estimate the colony titer a sample is plated on GAL/RAF/CM-ura-his-trp-leu plates. After determining the titer, library screening is continued by plating approximately 1.000.000 colonies on 10 cm/10 cm plates each. A total of 36.000.000 colonies is plated on leu selection plates GAL/CM-his-ura-trp-leu (20 million from vial A and 16 million from vial B). Colonies are isolated when the diameter of the colonies is at least 1\ mm. The numbers of isolated colonies from each day and vial are indicated in the table below:

| 2 days | 3 days | 4 days |
|---|---|---|
| 15A | 93A | 27A |
| 9B | 81B | 25B |

All isolated colonies are replated on different plates for determination of LacZ activity and only those colonies are selected which fit to the described criteria for each medium:

Numbers of isolated colonies from each day and vial are indicated:

| | | |
|---|---|---|
| GAL/RAF/CM | -ura-his-trp-leu | growth yes |
| GLU/CM | -ura-his-trp-leu | growth no |
| GAL/RAF/CM | -ura-his-trp + Xgal | blue and growth yes |
| GLU/CM | -ura-his-trp + Xgal | not blue, growth yes |

| <12 hours | 20 hours | 28 hours | 48 hours | 72 hours |
|---|---|---|---|---|
| 4A | 17A | 9A | 11A | 24A |
| 2B | 6B | 5B | 15B | 24B |

A total of approximately 250 colonies is growing on leucine selection plates and tested for lacZ activity. 107 of these colonies show blue staining as an indication for lacZ activity. Colony PCR performed on these 107 colonies with primers around the cloning site of the prey vector pJG4-5 generates approximately 10 different groups of CDNA clones based on PCR size. Sau3A1 digestion of the PCR fragments makes a more detailed grouping of different classes of SERK-interacting candidate CDNA clones possible. Members of all different classes are used to isolate and to clone the prey plasmid into E.coli and to determine the nucleotide and predicted amino acid sequence. Prey plasmids are retransformed in yeast and tested for SERK-dependent activation of leu selection and lacZ activity. All classes of CDNA clones prove to display a SERK-dependent yeast LexA two hybrid interaction after retransformation experiments. All these clones represent intracellular or membrane-attached factors involved in the signalling pathway mediated by the SERK receptor kinase protein. A total of 8 different classes of SERK-interacting proteins is identified.

### Example 2

### Function of SERK-interacting Proteins

Four of the classes of proteins that show an interaction with SERK are members of the family of Squamosa-promoter Binding Protein (SBP) transcription factors (Klein et at, Mol. Gen Genet 250:7-16, 1996). They are represented by the clones 3A35 (SEQ ID NO:1 and SEQ ID NO:2), 3B39 (SEQ ID NO:3 and SEQ ID NO:4), 4B19 (SEQ ID NO:5 and SEQ ID NO:6), and 3A52 (SEQ ID NO:7 and SEQ ID NO:8). These proteins are able to interact specifically with DNA through a conserved domain of 79 amino,,acid residues, the SBP-box. Within the SBP-box a remarkable arrangement of cysteine and histidine residues can be recognized, which is reminiscent of zinc-fingers and probably involved in the recognition of specific promoter elements. A bipartite nuclear localization signal is placed at the C-terminal end of the SBP-box (Dingwall et al, Trends Biochem Sci 16:478-481, 1991). Both the N-terminal and the C-terminal domains of the SERK-interacting SBP proteins are highly variable and are probably involved in regulation of protein activity. One of the classes of SBP proteins, represented by 4B19, is identical with SPL3, a gene involved in the floral transition and expressed in developing flower buds (Cardon and Hohmann 1997 Plant Journal 12, 367-377). The most likely model for the signaling pathway mediated by the SERK and SBP proteins is transphosphorylation of cytoplasmic SBP-transcription factors by SERK after ligand binding, followed by nuclear translocation of the factors and binding to specific regulatory DNA target sites on the genome. A similar mode of signal transduction has been described for animal serine-threonine receptor-kinase proteins which are known to transphosphorylate a family of so called SMAD transcription factors. Phosphorylated activated SMAD proteins are translocated into the nucleus (Heldin et al, Nature 390:465-471, 1997).

Another class of SERK-interacting proteins is represented by an isoform of the family of 14-3-3 proteins. 4B11 (SEQ ID NO:9 and SEQ ID NO:10) is identical to the 14-3-3 type lambda protein (Wu et al, Plant Physiol 114:1421-1431, 1997). A total of 10 different 14-3-3 proteins is present in Arabidopsis and the different members are involved in intracellular signal transduction. They mediate signal transduction by binding to phosphoserine-containing proteins on specific binding motifs represented by conserved amino acid sequences like RxxS(p)xP (Yaffe et al, Cell 91:961-971, 1997). A putative 14-3-3 interaction domain having the amino acid sequence RPPSQP is also found at position 391-396 of the Arabidopsis SERK protein, and at the corresponding aligned region of the Daucus carota SERK protein having the amino acid sequence RQPSEP providing SERK with a mechanism for a 14-3-3 mediated signal transduction.

4A24 (SEQ ID NO:11 and SEQ ID NO:12) represents a member of a small new Arabidopsis gene family from which one member has already been described in the literature as the NDR1 protein (Century et al, Science 278:1963-1965, 1997). NDR1 is likely to encode a membrane-associated component in the signal transduction pathway downstream of pathogen-recognizing proteins. It was suggested that NDR1 is a protein that interacts with many different receptors to transduce their signal. 4A24 represents a new member in this small family of proteins and might have an important function in intracellular signal transduction mediated by transmembrane receptors.

Clone 3B76 (SEQ ID NO:13 and SEQ ID NO:14) encodes a protein with homology to a domain in E.coli aminopeptidase N. and might encode an Arabidopsis protease, interacting or activated by SERK.

The predicted amino acid sequence represented by clone 4A5 (SEQ ID NO:15 and SEQ ID NO:16) has no homology with known gene products although there is small not yet described family of related gene products in Arabidopsis (AA585806, AA651106, T45539).

### Example 3

### Transformation of Arabidopsis with Genes Encoding SERK-interacting proteins

Plasmids containing promoter sequences

The CaMV 35S promoter enhanced by duplication of the −343 to −90 region (Kay et al, Science 236:1299-1302, 1987) is isolated from the mMON999 vector by digestion with HindIII and SstI and cloned into the pBluescript SK-vector resulting in vector pMT120.

The promoter of the FBP7 gene from Petunia (Angenent et al, Plant Cell 7: 1569-1582, 1995) is cloned by subcloning the 0.6 kb HindIII-XbaI genomic DNA fragment of FBP7 into the HindIII-XbaI site of pBluescript KS-resulting in the vector FBP201.

Plasmids containing full length SERK-interacting cDNA clones

Full length cDNA of the identified SERK-interacting gene products is produced by RT-PCR amplification of early stage Arabidopsis silique RNA. Full length cDNA is isloated from clones 3A35, 3A52, and 4B19, Clone 3B39 16 was already present as a full length cDNA clone. Oligo sequences are based on the nucleotide sequences from identical BAC or EST clones.

Binary vector constructs

Based on the pBIN19 vector, a binary vector is constructed for transformation of the Arabidopsis thaliana SERK-interacting cDNA under the control of different promoters. The full length cDNA clones of the putative SBP-transcription factors interacting with SERK are blunted by Klenow treatment and cloned into the Smal site of pBIN19. The polyadenylation sequence from the pea rbcS::E9 gene (Millar et al, Plant Cell 4:1075-1087, 1992) is placed downstream from the coding sequence by cloning a Klenow-filled EcoRI-HindIII E9 DNA fragment into the Klenow-filled XmaI site of the pBIN19:SERK interacting factor in order to generate the binary vectors pAt3A35, pAt3A52, pAt4B19 and pAt3B39. The pAt binary vectors are used to generate promoter-SERK interacting factor constructs.

The CaMV 35S promoter is cloned in the SmaI site of the pAT vector constructs as a Klenow-filled KpnI-SstI fragment to give p35SAt vectors.

The Sacl-Kpnl fragment of FBP201 is filled with Klenow and cloned into the SmaI site of the pAt vector constructs to give the pFBP201At vectors.

Introduction of plant expression vectors into Arabidopsis thaliana plants

The above described vector constructs are electrotransformed into Agrobacterium tumifacienses strain C58C1. Wild type Arabidopsis thaliana WS plants are grown under standard long day conditions (16 hours light and 8 hours dark). The first emerging influorescence is removed in order to increase the number of influorescences. Five days later, plants are used for the vacuum infiltration procedure. Transformed Agrobacterium C58C1 is grown on LB plates with 50 mg/l kanamycin, 50 mg/l rifampicin and 25 mg/l gentamycin. Single colonies are used to inoculate 500 ml of liquid medium (as described above) and grown O/N at 28° C. Log phase culture (OD_{600=0.8}) is centrifuged to pellet cells and resuspended in 150 ml of infiltration medium (0.5 ×MS medium pH 5.7, 5% sucrose and 1 mg/l benzylaminopurine). The influorescences of 6 Arabidopsis plants are submerged in the infiltration suspension while the remaining parts of the plants (which are still potted) are placed upside down on meshed wire to avoid contact with the infiltration medium. Vacuum is applied to the whole set-up for 10 min at 50 kPa. Plants are directly afterwards placed under standard long day conditions. After completed seed setting the seeds are surface sterilized by an 1 % sodium hypochlorite soak, thoroughly washed with sterile water and planted onto petridishes with 0.5×MS medium, 1% agar and 80 mg/l kanamycin in order to select for transformed seeds. After 7 days of germination under long day conditions (10.000 lux) the transformed seedlings can be identified by their green color of their cotyledons and the appearance of the first true leaves. Transformed seedlings are further grown in soil under long day conditions. The vacuum infiltration method results in approximately 0.1% transformed seeds.

What we claim is:

1. A method for increasing the probability of vegetative reproduction of a new plant generation comprising transgenically expressing a gene encoding a protein acting in the signal transduction cascade triggered by the Somatic Embryogenesis Receptor Kinase (SERK).

2. A method according to claim 1, wherein the encoded protein physically interacts with SERK.

3. The method according to claim 2, wherein the protein is a member of the family of Squamosa-promoter Binding Protein (SBP) transcription factors or 14-3-3 type lambda proteins.

4. The method according to claim 2, wherein the protein has the amino acid sequence given in SEQ ID NO:2, SEQ ID NO:4, SEQ ID NO:6, SEQ ID NO:8, SEQ ID NO:10, SEQ ID NO:12, SEQ ID NO:14, or SEQ ID NO:16, or an amino acid sequence having a component sequence of at least 150 amino acids length which after alignment reveals at least 40% identity with SEQ ID NO:12 or SEQ ID NO:16.

5. The method according to claim 1 increasing the probability of vegetative reproduction through seeds (apomixis).

6. The method according to claim 5, wherein the seeds result from non-gametophytic apomixis.

7. The method according to claim 5, wherein the encoded protein is transgenically expressed in the vicinity of the embryo sac.

8. The method according to claim 1 increasing the probability of in vitro somatic embryogenesis.

9. The method according to claim 1, wherein expression of the gene is under control of the SERK gene promoter, the carrot chitinase DcEP3-1 gene promoter, the Arabidopsis AtChitIV gene promoter, The Arabidopsis LTP-1 gene promoter, The Arabidopsis bel-1 gene promoter, the petunia fbp-7 gene promoter, the Arabidopsis ANT gene promoter or the promoter of the O126 gene of Phalaenopsis.

10. A gene encoding a protein having the amino acid sequence given in SEQ ID NO:2, SEQ ID NO:4, SEQ ID NO:6, SEQ ID NO:8,SEQ ID NO:10, SEQ ID NO:12,SEQ ID NO:14, or SEQ ID NO:16, or an amino acid sequence having a component sequence of at least 150 amino acids length which after alignment reveals at least 40% sequence identity with SEQ ID NO:12 or SEQ ID NO:16.

11. A gene according to claim 10 having the nucleotide sequence given in SEQ ID NO:1, SEQ ID NO:3, SEQ ID NO:5, SEQ ID NO:7, SEQ ID NO:9, SEQ ID NO:11, SEQ ID NO:13, or SEQ ID NO:15.

12. A gene according to claim 10 wherein the nucleotide sequence is modified in that known mRNA instability motifs or polyadenylation signals are removed and/or codons which are preferred by the plant into which the DNA is to be inserted are used.

13. A plant or plant cell transgenically expressing the gene according to any one of claims 10-12.

14. A plant or plant cell obtainable by the method of claim 1.
